(19) United States
(12) Patent Application Publication
(10) Pub. No.: US 2001/0006673 A1
(43) Pub. Date: Jul. 5, 2001

(54) LUTEIN- CONTAINING SUPPLEMENT AND PROCESS FOR ENHANCING IMMUNE RESPONSE IN ANIMALS

(76) Inventor: MICHAEL G. HAYEK, DAYTON, OH (US)

Correspondence Address: KILLWORTH GOTTMAN HAGAN & SCHAEFF LLP, ONE DAYTON CENTRE, ONE SOUTH MAIN STREET SUITE 500, DAYTON, OH, 454022023

(21) Appl. No.: 09/291,227

(22) Filed: Apr. 13, 1999

Related U.S. Application Data
(60) Provisional application No. 60/081,968, filed on Apr. 16, 1998.

(51) Int. Cl. A61K 9/68

(52) U.S. Cl. 424/442; 424/440

(57) ABSTRACT

A food supplement and process for feeding a companion animal such as a dog or cat a diet containing an effective amount of lutein to enhance immune response and improve the overall health of the animal is provided. Preferably, the diet includes from about 1 to about 50 mg/day of lutein (from about 2 to about 315 mg lutein/kg diet). Such a diet provides sufficient lutein to be absorbed by the animal and supplied by the blood and blood leukocytes and neutrophils in the animal.

### CROSS REFERENCE TO RELATED APPLICATIONS

This application claims the benefit of U.S. provisional application Ser. No. 60/081,968, filed Apr. 16, 1998.

### BACKGROUND OF THE INVENTION

This invention relates to a pet food supplement and process for enhancing immune response and improving the overall health of companion animals such as cats and dogs, and more particularly, to a pet food supplement and process which includes beneficial amounts of lutein in the animal's diet.

Carotenoids are naturally-occurring plant pigments which are absorbed in varying degrees by different species. Common carotenoids include β-carotene, lycopene, astaxanthin, and canthaxanthin. These carotenoids are known to play an important role in modulating the immune system and enhancing the health of these species. For example, canthaxanthin has been found to prevent chemical-induced carcinogenesis in mice while α-carotene has been found to inhibit human neuroblastoma cell proliferation. Canthaxanthin has also been found to increase lymphocyte proliferation in rats and enhance the production of tumor necrosis factor (TNF) by macrophages in hamsters. Astaxanthin and β-carotene have been found to increase ex vivo antibody response of mouse splenocytes to T-dependent antigens. However, the majority of studies to date have focused on the effects of β-carotene.

Few studies have been conducted with regard to the physiologic function of lutein, a major blood carotenoid in some species (human, chicken). In some recent studies, it has been found that dietary lutein decreases mammary tumor incidence, tumor growth and has shortened tumor latency in mice challenged with a transplantable murine mammary tumor cell line. Dietary lutein was also found to enhance lymphocyte proliferation in mouse splenocytes. Splenocytes from mice injected peritoneally with lutein, astaxanthin, and β-carotene have shown enhanced antibody response to T-dependent antigens. Therefore, carotenoids including lutein possess immunomodulatory activities and may be important in disease etiology.

Accordingly, there is still a need in the art for promoting a healthy immune system in companion animals such as dogs and cats.

### SUMMARY OF THE INVENTION

The present invention meets that need by providing a process for feeding a companion animal such as a dog or cat a diet containing an effective amount of lutein to enhance immune response and improve the overall health of the animal. Preferably, the animal is fed a diet which includes from about 1 to about 50 mg/day of lutein (from about 2 to about 315 mg lutein/kg diet). Such a diet provides sufficient lutein to be absorbed by the animal and supplied to the blood and blood leukocytes and neutrophils in the animal.

The lutein is preferably provided in a dietary supplement for the animal which comprises, on a dry matter basis, from about 0.001 to about 2% by weight lutein. Where the companion animal is a dog, the supplement preferably comprises from about 0.01% to about 1% by weight lutein. Where the companion animal is a cat, the supplement preferably comprises from about 0.01% to about 1% by weight lutein.

Accordingly, it is a feature of the present invention to provide a pet food supplement and process for enhancing immune response and improving the overall health of companion animals such as dogs and cats by providing an effective amount of lutein in the diet of the animal. This, and other features and advantages of the present invention, will become apparent from the following detailed description, the accompanying drawings, and the appended claims.

### BRIEF DESCRIPTION OF THE DRAWINGS

FIG. 1 is a graph of concentration of blood plasma lutein in dogs (nmol/L) versus time;

FIG. 2 is a graph of DTH response in dogs fed lutein daily for 6 weeks;

FIG. 3 is a graph of DTH response in dogs fed lutein daily for 12 weeks;

FIG. 4 is a graph of PHA-stimulated PBMC response in dogs fed lutein daily for 12 weeks;

FIG. 5 is a graph of Con A-stimulated PBMC response in dogs fed lutein daily for 12 weeks;

FIG. 6 is a graph of PWM-stimulated PBMC response in dogs fed lutein daily for 12 weeks;

FIG. 7 is a graph of the effect of dietary lutein on the percentages of CD4+ T-helper cells and CD8+ T-cytotoxic cells in dogs fed lutein daily for 12 weeks;

FIG. 8 is a graph of the effect of dietary lutein on the percentages of CD5+ cells and MHC II+ cells in dogs fed lutein daily for 12 weeks;

FIG. 9 is a graph of concentrations of plasma polyclonal IgG and IgM in dogs fed lutein daily for 17 weeks;

FIG. 10 is a graph of concentration of blood plasma lutein in cats (μmol/L) versus time;

FIG. 11 is a graph of Con A-stimulated PBMC response in cats fed lutein daily for 12 weeks;

FIG. 12 is a graph of PWM-stimulated PBMC response in cats fed lutein daily for 12 weeks;

FIG. 13 is a graph of DTH response in cats fed lutein daily for 6 weeks;

FIG. 14 is a graph of DTH response in cats fed lutein daily for 12 weeks;

FIG. 15 is a graph of the effect of dietary lutein on the percentages of CD3+ T cells and CD21+ B cells in cats fed lutein daily for 12 weeks;

FIG. 16 is a graph of the effect of dietary lutein on the percentages of CD4+ T-helper cells and CD8+ T-cytotoxic cells in cats fed lutein daily for 12 weeks; and

FIG. 17 is a graph of concentrations of plasma polyclonal IgG in dogs fed lutein daily for 12 weeks.

### DETAILED DESCRIPTION OF THE PREFERRED EMBODIMENTS

The present invention uses a pet food composition which contains a source of lutein as a supplement in an amount between about 1 to 50 mg/day of lutein (from about 2 to about 315 mg lutein/kg diet). Such a diet provides sufficient lutein to be absorbed by the animal and supplied to the blood and blood leukocytes and neutrophils in the animal. Both domestic dogs and cats are able to absorb dietary lutein. Furthermore, circulating lutein is significantly absorbed by both peripheral blood lymphocytes and neutrophils in such animals. Lutein is also distributed in the various subcellular organelles. Such lutein in the various organelles of leukocytes is believed to (1) protect these cells from oxygen free radical attack and/or (2) directly regulate nuclear events. Thus, feeding companion animals such as dogs and cats effective amounts of lutein provides lutein at important cellular sites in the body tissues of the animal which results in an up-regulation of immune function and improved health in such animals.

The pet food composition can be any suitable pet food formula which also provides adequate nutrition for the animal. For example, a typical canine diet for use in the present invention may contain about 20 to 40% crude protein, about 4 to 30% fat, and about 4 to 20% total dietary fiber. However, no specific ratios or percentages of these or other nutrients are required. The lutein may be blended with such pet food to provide the beneficial amounts needed.

In order that the invention may be more readily understood, reference is made to the following examples which are intended to illustrate the invention, but not limit the scope thereof.

### EXAMPLE 1

### Lutein Uptake in Dogs

Fifty six female Beagle dogs (17 to 18 months old; average body weight 11.4±0.4 kg) were randomly assigned to be supplemented daily with 0, 5, 10 or 20 mg lutein for 17 weeks. The lutein contained 76.66% lutein and 5.23% zeaxanthin. The lutein supplement was resuspended in soybean oil to the appropriate concentration and 1 mL administered perorally at 0800 h daily. Food (200 g/dog/d) was offered immediately after the lutein supplementation. The basal diet shown in Table 1 below met or exceeded the requirements for all essential nutrients (NRC 1985).

TABLE 1

| Component | Composition (g/kg) |
|---|---|
| Moisture | 66 |
| Protein | 262 |
| Ash | 74 |
| Fat | 160 |
| Calcium | 14.8 |
| Phosphorus | 10.3 |
| Gross Energy (kcal/kg) | 4783 |

All dogs were housed in 2×2 m pens (2 dogs/pen) in a temperature (20 to 22° C.) and light (14 h light) controlled facility. Body weight was recorded at weeks 0, 6, and 12.

Blood was collected by jugular venipuncture into heparinized evacuated tubes on weeks 0, 2, 4, 8 and 12 and aliquots used for HPLC analysis and for assessing immune responses.

### Extraction and HPLC Analysis

Plasma was extracted for analysis of lutein, zeaxanthin, retinol and α-tocopherol. Briefly, plasma protein was precipitated by adding an equal volume of ethanol containing 0.1% butylated hydroxytoluene (BHT) (Aldrich Chemical Co., Milwaukee, Wis.). The mixture was extracted with 5 mL of a 1:1 mixture of petroleum ether:anhydrous diethyl ether.

The dried residue was resuspended in mobile phase consisting of a 47:47:6 (v:v:v) mixture of HPLC-grade acetonitrile: methanol: chloroform (Fisher Scientific, Fair Lawn, N.J.). Lutein, zeaxanthin and α-tocopherol were quantitated by comparing the area under the curve whereas retinol was quantitated using peak height. The identity of the eluted compounds was confirmed by comparing their absorption spectrum with that of pure standards. Because baseline separation of lutein and zeaxanthin was not accomplished, plasma concentrations were reported as lutein+zeaxanthin.

### Delayed Type Hypersensitivity Response

Skin induration response was assessed in all dogs on weeks 0, 6, and 12. Dogs were injected intradermally in the flank area with saline (8.5 g/L; control), an attenuated polyvalent vaccine containing canine distemper virus, canine adenovirus type-2, canine parainfluenza virus and parvovirus (Vanguard 5, Smithkline Beacham, West Chester, Pa.; specific antigen), and PHA (0.5 g/L; nonspecific antigen). The doses of vaccine and PHA used were previously determined to provide optimal skin response in Beagle dogs of similar age. The injection site was clipped and wiped with 70% ethyl alcohol. The injection volume was 100 μL. Skin induration was measured at 0, 24, 48 and 72 h post-injection with the aid of a pressure-sensitive digital micrometer (Mitsutoyo, Tokyo, Japan) and response was expressed as a percentage of skin thickness measured at 0 h.

### Lymphocyte Proliferation

Blood collected on weeks 0, 2, 4, 8 and 12 were used to assess mitogen-induced lymphocyte proliferation by peripheral blood mononuclear cells (PBMC). Whole blood culture was used in order to mimic in vivo conditions. Mitogens used were phytohemagglutinin (PHA), concanavalin A (Con A) and pokeweed mitogen (PWM). Whole blood was thoroughly mixed and then diluted 1:12 with RPMI-1640 containing 25 mM of Hepes, penicillin (100 U/mL) and streptomycin (100 μg/mL) (Sigma, St. Louis, Mo.). Preliminary studies using undiluted blood and blood diluted 1:2, 1:4, 1:8, 1:12 and 1:16 showed optimal PBMC proliferative response with a 1:12 dilution. Triplicate 150 μL volumes were pipetted into 96-well round bottom plates and 50 μl of the appropriate mitogens added. The final concentrations of the mitogens in culture were 2 and 10 μg/mL for PHA, 1 and 5 μg/mL for Con A and 0.5 and 2.5 μg/mL for PWM. The two mitogen concentrations gave maximum (the higher mitogen concentration) and suboptimal (lower mitogen concentration) proliferative responses in preliminary studies using blood from similar animals. The mixture was incubated for 72 h at 37° C. in a humidified incubator under a 5% CO₂ atmosphere. Four hours prior to the termination of the incubation period, 20 μL of [³H]-thymidine (1 [μCi/well) were added. Cells were harvested onto fiberglass filters and radioactivity was counted by liquid scintillation. Proliferation response of PBMC was expressed as stimulation index (cpm of stimulated cultures/cpm of unstimulated cultures).

### Lymphocyte Subsets

Blood leukocytes were separated using Histopaque-1119 (Sigma, St. Louis, Mo.). Cells were washed three times with phosphate-buffered saline (PBS, pH 7.4) and contaminating erythrocytes were lysed in NH₄CI (8.4 g/L). Lymphocyte subsets were determined by flow cytometry (FACScan, Becton Dickinson, San Jose, Calif.). Isolated mononuclear cells were resuspended to 1×10⁷ cells/mL in PBS supplemented with 2% gamma globulin-free serum, 5% goat serum and 0.2 g/L sodium azide. For immunofluorescence analysis, a total of 5×10⁵ cells were incubated for 30 minutes on ice with optimal concentrations of mouse anti-canine monoclonal antibody (mAb). The mAb used were specific for the following lymphocyte subsets: total T cells (anti-CD5), T-helper cells (anti-CD4), T-cytotoxic/suppressor cells (anti-CD8), lymphocytes expressing major histocompatability complex (MHQ class II antigens (anti-MHC class II), and B cells (anti-CD21). Cells were then washed three times and incubated with a secondary antibody, fluorescein isothiocyanate (FITC)-conjugated goat F(ab′)₂ anti-mouse IgG+IgM (H+L) (Caltag, Burlingame, Calif.), for 30 minutes on ice to visualize the bound mAb. Stained cells were fixed in 4% paraformaldehyde in preparation for acquisition.

Appropriate negative controls were included to correct for background fluorescence. Data were expressed as the percentage of positive-staining cells corrected for cells stained nonspecifically with the secondary antibody.

### NK Cell Cytotoxicity

Canine thyroid adenocarcinoma cells were used as target cells in assessing NK cell cytotoxic activity. This cell line was previously shown to be susceptible to killing by canine NK cell. Target cells were cultured in a T75 flask with 20 mL minimum essential medium (MEM; Sigma Chem. Co., St. Louis, Mo.) supplemented with 10% fetal calf serum (FC S), 100 U/mL of penicillin and 100 μg/mL of streptomycin. When confluent, target cells were trypsinized, washed 3 times and resuspended to 5×10⁵ cells/mL in complete medium (RPMI-1640+10% FCS+100 U/mL of penicillin+100 μ/mL of streptomycin). Triplicate 100 μL aliquots of the target cells were pipetted into 96-well U-bottom plates (Costar, Cambridge, Mass.) and incubated for 8 hours to allow cell adherence. Lymphocytes (effector cells; 100 μL) isolated by percoll separation (as described above) were then added to the target cells to provide an effector:target cell (E:T) ratio of 10:1. After 10 hours of incubation at 37° C., 20 μl of a substrate containing 5 μg of 3-(4,5-dimethylthiazol-2-yl)-2,5-diphenyltetrazolium bromide (MTT) was added. The mixture was incubated for 4 hours at 370° C. after which the unmetabolized MTT was removed by aspiration. The formazan crystals were dissolved by adding 200 μL of 95% ethanol. Optical density was measured at 570 nm using a microplate reader. The percentage of NK cell-specific lysis was calculated as follows:

Specific cytotoxicity (%)=100×{1−[(OD of target cells and effector cells−OD of effector cells)/(OD of target cells)]}

### IL-2 Production

Whole blood was diluted 1:2 with RPMI-1640 supplemented with Hepes and antibiotics (described earlier) and 400 μL of diluted blood were pipetted into 48-well plates (Costar, Cambridge, Mass.). Cells were stimulated with PHA (400 μL of a 5 μg/mL solution) for 48 hours at 370° C. in a 5% CO₂ atmosphere. The plates were centrifuged at 200×g for 10 minutes and the supernatant was frozen at −80° C. The IL-2 content of the culture supernatant was determined in triplicate by ELISA (Intergen, Purchase, N.Y.). The polyclonal anti-human IL-2 cross-reacted with canine IL-2 and recombinant human IL-2 was used as the standard.

### Serum IgG and IgM

Serum collected on weeks 0, 2, 4, 8 and 12 were analyzed for IgG and IgM concentrations by single radial immunodiffusion (SRID). In addition, all dogs were vaccinated with the polyvalent vaccine (Vanguard 5, Smithkline Beacham, West Chester, Pa.) on week 13 and again on week 15 to study the possible anamnestic effect of dietary lutein. Blood was collected weekly from week 13 through week 17. Goat antiserum to canine IgG (10%, whole molecule) or IgM (7.5%, μ chain specific) (ICN, Aurora, Ohio) was mixed with melted agarose (10 g/L in PBS; Sigma Chem. Co., St. Louis, Mo.) and the mixture solidified in SRID plates.

Duplicate 5 μL volumes of serum or IgG (0, 2.88, 5.75, 11.5 and 23.0 mg/ml) or IgM (0, 0.25, 0.5, 1.0 and 2.0 mg/ml) standard were loaded into the wells. After incubating for 24 hours at room temperature in a humidified chamber, ring diameters were measured using a SRID reader (Transidyne General Corp., Ann Arbor, Mich.).

### Lipid Peroxidation (TBARS) Assay

Plasma lipid peroxidation activity was determined by measuring malondialdehyde (MDA) production. The standard used was 1,1,3,3-tetramethoxypropane JMP) (Sigma Chem. Co., St. Louis, Mo.). Duplicate 500 μL plasma samples were pipetted into 15 ml, glass tubes and 3 mL of 10 g/L phosphoric acid and 1 ml of 6 g/L TBA solution were added. The mixture was boiled in a water bath for 45 minutes. Tubes were allowed to cool and the TBA-MDA complex was extracted with 4 ml of n-butanol. The butanol layer was separated by centrifugation at 1,000×g for 10 minutes and absorbance was measured on at 532 nm (Beckman, Fullerton, Calif.). The TBARS activity was expressed in nmole MDA/L plasma.

### Statistics

Data were analyzed by split-plot ANOVA using the General Linear Model of SAS. The statistical model was Y_{yk}=μ+Dietᵢ+Dogⱼ(Diet) (error term used to test the effects of Diet)+Periodₖ+Dietᵢ*Periodₖ+e_{yk}. Differences among treatment means were compared using orthogonal contrast and were considered statistically significant when P<0.05.

### RESULTS

### Body weight

Dietary lutein did not significantly affect changes in body weight during the experimental period as shown below in Table 2. Body weight across all diets and periods averaged 11.4±0.4 kg.

TABLE 2
Average body weight (kg) of dogs supplemented daily with
0, 5, 10 or 20 mg lutein¹ Dietary lutein, mg/d

| Week | 0 | 5 | 10 | 20 |
|---|---|---|---|---|
| 0 | 11.7 ± 0.3 | 11.2 ± 0.4 | 11.6 ± 0.5 | 11.2 ± 0.3 |
| 6 | 11.0 ± 0.3 | 11.0 ± 0.3 | 11.5 ± 0.3 | 10.9 ± 0.3 |
| 12 | 12.1 ± 0.4 | 11.7 ± 0.4 | 11.8 ± 0.4 | 11.6 ± 0.3 |

¹Values are means ± SEM (n = 14/diet/period). There was no significant treatment difference in body weight within a sampling period as analyzed by repeated measures ANOVA.

### Plasma

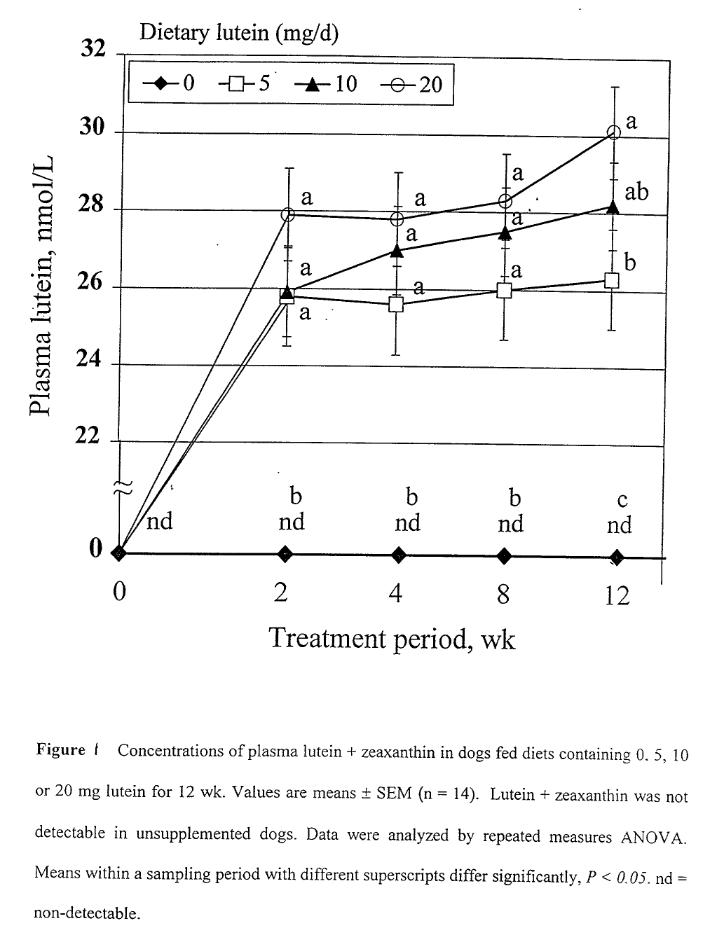

Plasma concentrations of lutein+zeaxanthin in lutein-supplemented dogs increased rapidly after 2 weeks of feeding (FIG. 1). Plasma concentrations continued to increase thereafter, although more gradually, in dogs fed 10 and 20 mg lutein. In contrast, plasma lutein+zeaxanthin was not detectable in unsupplemented dogs. Concentrations of plasma lutein+zeaxanthin on weeks 2 through 12 was significantly higher (P<0.05) in dogs fed lutein than in unsupplemented animals. Lutein supplementation did not influence concentrations of plasma retinol and α-tocopherol. Concentrations of these vitamins across all treatments and all periods averaged 3.63±0.14 and 37±2 μmol/L, respectively.

### Delayed-Type Hypersensitivity Response

During all periods studied, DTH response to saline was low (3 to 10% increase in skin thickness) and did not differ significantly among treatment groups. Prior to lutein feeding (week 0), DTH responses to PHA and vaccine were similar across all dietary groups. Irrespective of treatment period, maximal DTH response to PHA was observed around 24 h post-injection whereas maximal response to vaccine occurred between 48 and 72 h. Also, skin thickness response to PHA was approximately two times higher than to vaccine.

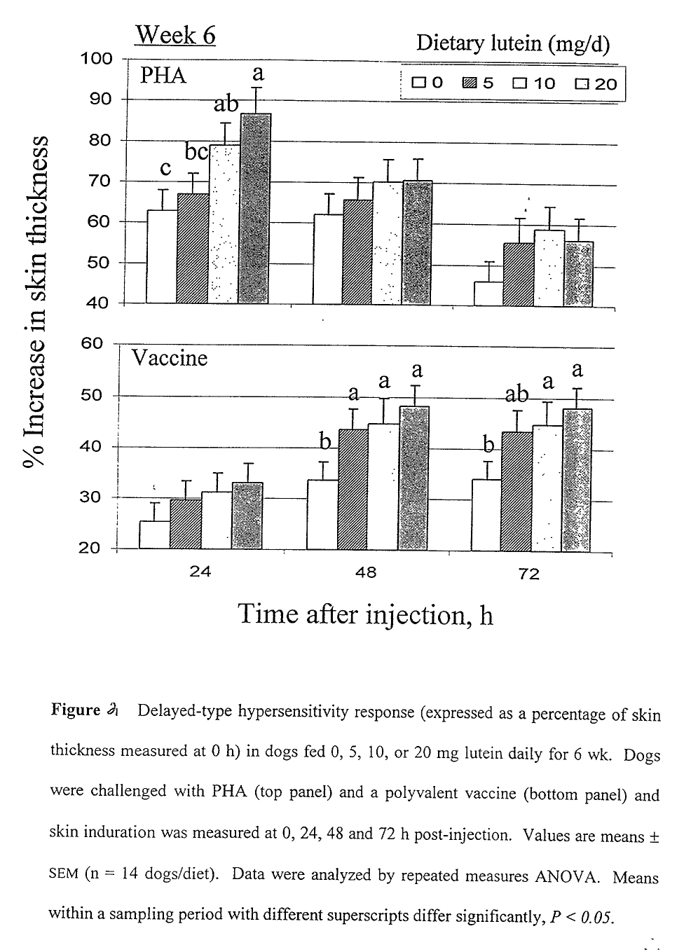

On week 6, there was a dose-related DTH response to PHA at 24 h post-injection as shown in FIG. 2. The DTH response decreased by 48 and 72 h and no significant treatment difference was observed during these times. The DTH response to vaccine was dose-related at 48 and 72 h post-injection and was not significant at 24 h.

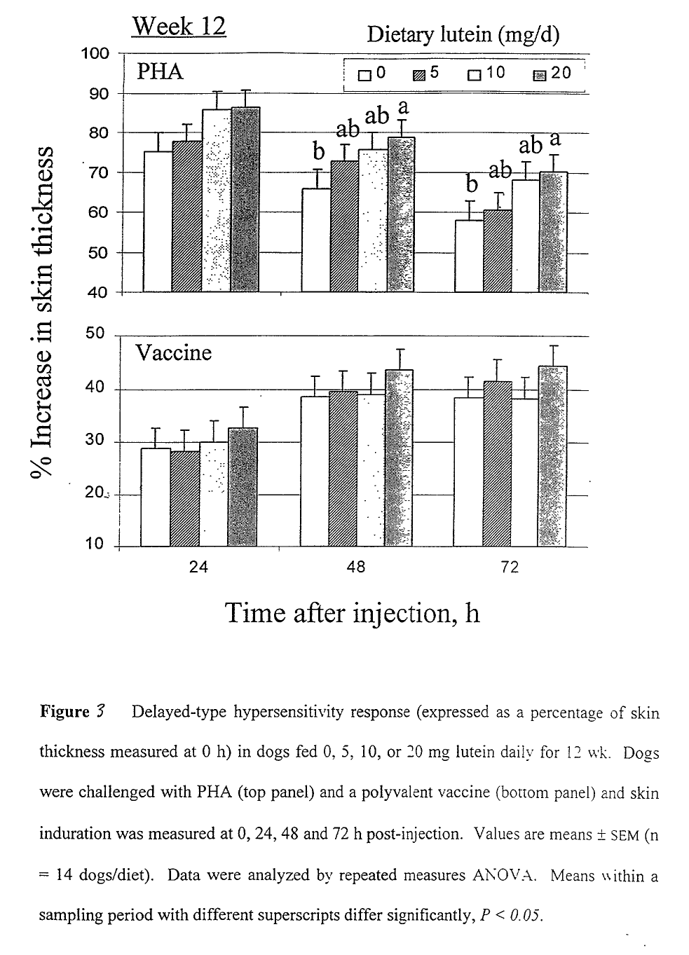

On week 12, there was a general dose-dependent increase in DTH response to PHA at 24, 48 and 72 h as shown in FIG. 3; however, the response was significantly higher for dogs fed 20 mg lutein compared to unsupplemented dogs. In contrast to week 6, there was no significant dietary effect on DTH response to vaccine.

### Mitogen-Induced PBMC Proliferation

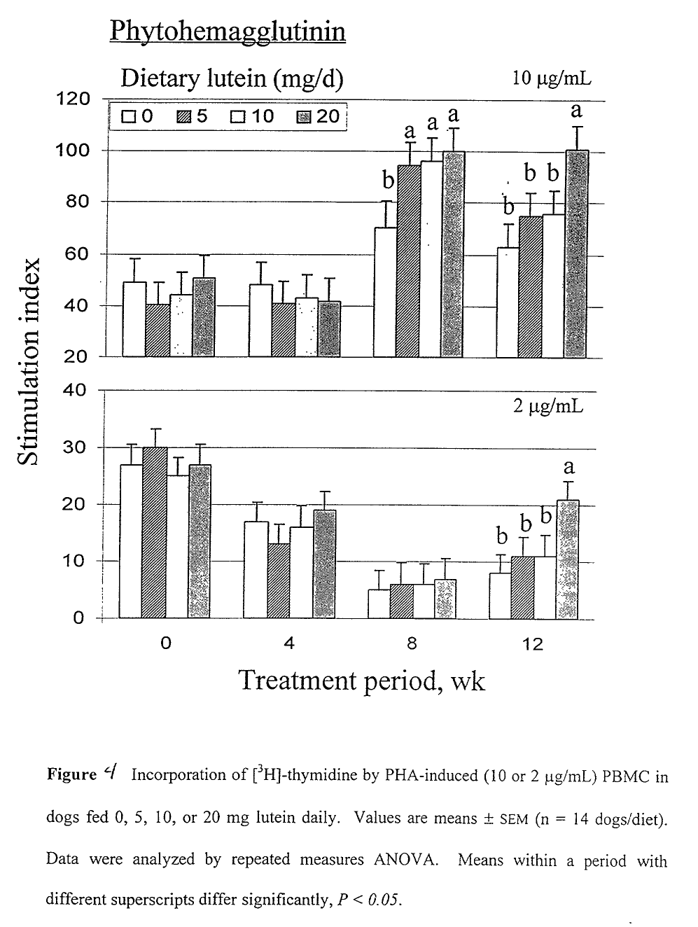

Dietary lutein did not significantly influence spontaneous proliferation by unstimulated PBMC. There was no significant effect of dietary lutein on PHA-stimulated PBMC response on weeks 0 and 4 as shown in FIG. 4. However, proliferative response was enhanced (P<0.01) on weeks 8 (10 pg/mL PHA) and 12 (2 and 10 μg/mL PHA) in dogs fed 20 mg lutein compared to unsupplemented dogs. Dogs fed 5 and 10 mg lutein also had higher PBMC proliferation on week 8 in response to 10 μg/ml PHA.

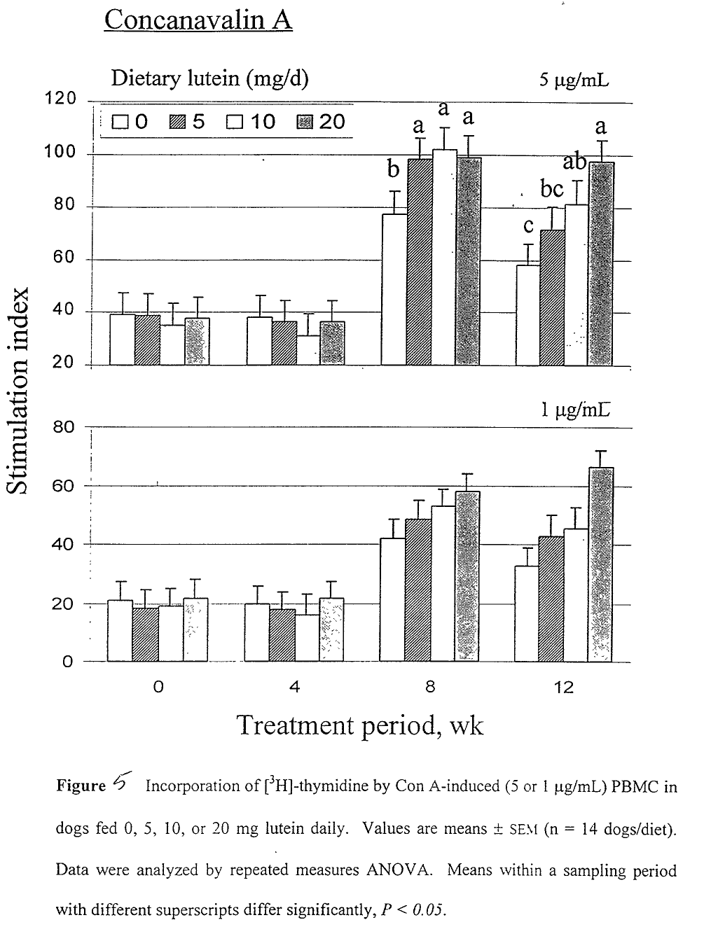

The effects of dietary lutein on Con A-stimulated PBMC proliferative response (see FIG. 5) were generally similar to those observed with PHA-induced proliferation. On weeks 8 and 12, dogs fed 20 mg lutein had higher PBMC proliferation in response to both concentrations of Con A. Dogs fed 10 mg lutein also showed higher PBMC proliferation on weeks 8 and 12 in response to 5 μg/mL Con A. Generally, PBMC proliferation was higher with 5 μg/mL than with 1 μg/mL Con A.

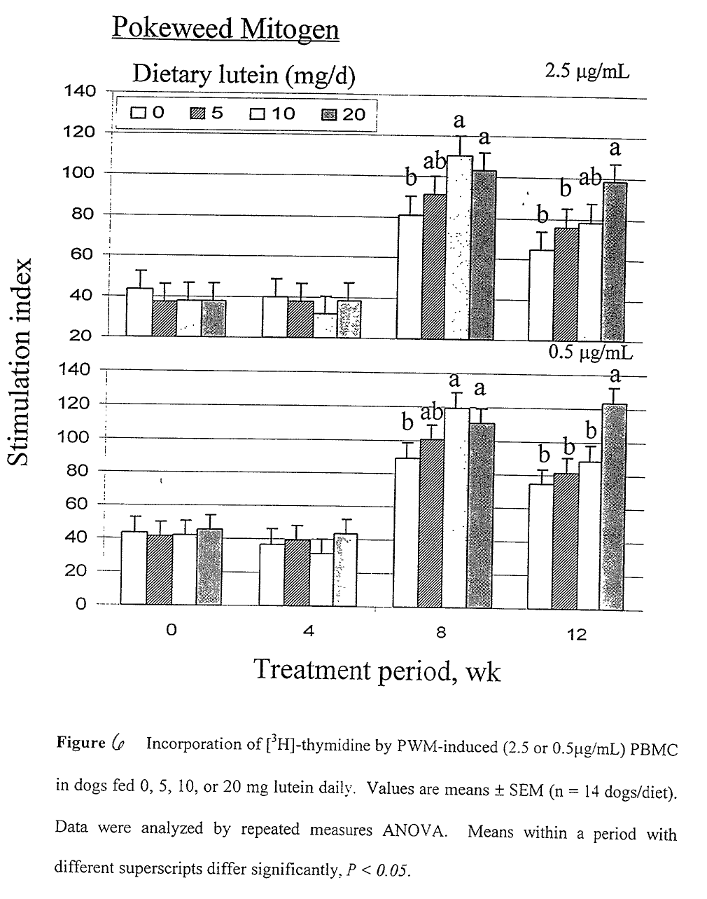

Proliferation of PBMC in response to PWM (FIG. 6) was generally similar to those observed with PHA and Con A. Dogs fed 20 mg lutein had significantly higher PBMC proliferation on week 8 and 12 in response to both concentrations of PWM as compared to unsupplemented controls. Those fed 10 mg lutein also had higher proliferative response on week 8. Again, no significant treatment difference was observed at weeks 0 and 4.

### Natural Killer Cell Cytotoxic Activity

Dietary lutein supplementation did not significantly influence NK cell cytotoxic activity. Specific lysis of target cells by PBMC averaged 49.1±1.1% across all treatments and sampling periods.

### Lymphocyte Subpopulations

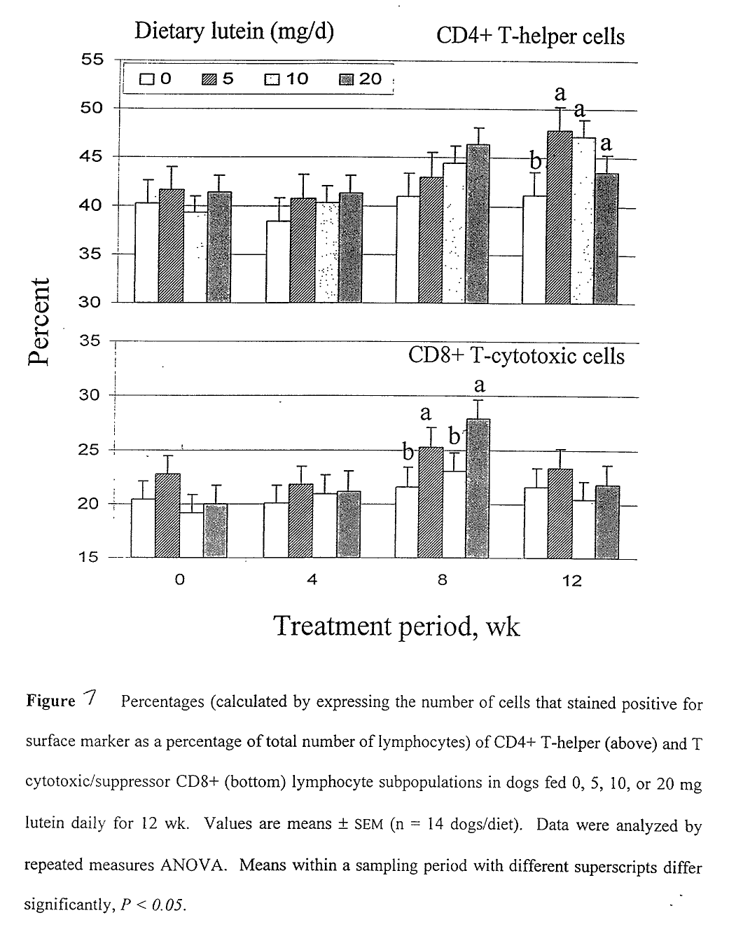

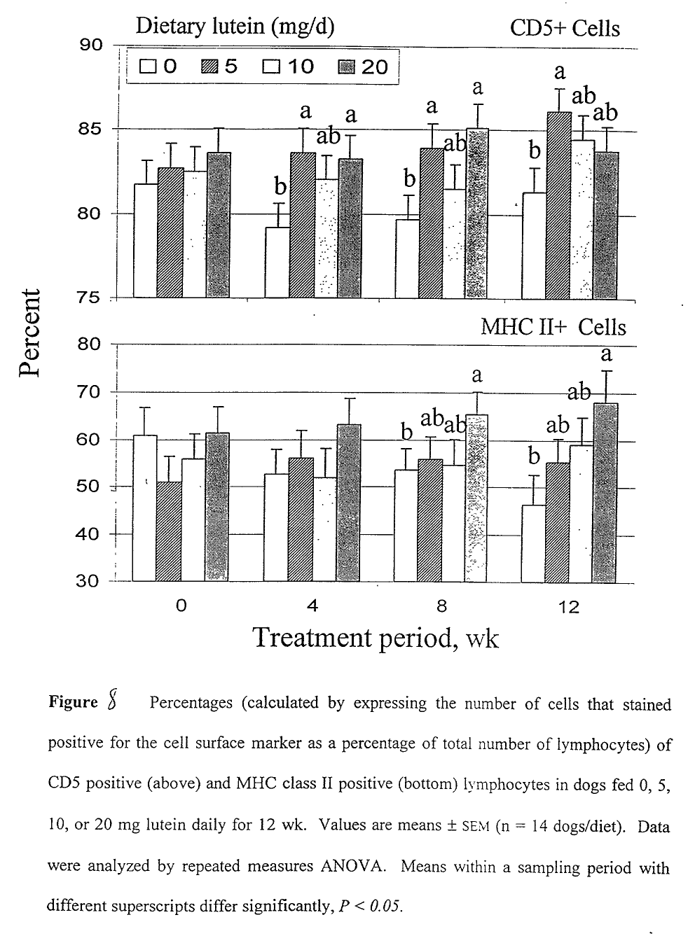

The effects of dietary lutein on the percentages of CD5+, CD4+, CD8+, CD21+ (B cells) and MHC class 11 lymphocyte subpopulations are illustrated in FIGS. 7 and 8. Prior to dietary lutein supplementation (week 0), there were no significant differences in the percentages of any of the lymphocyte markers. On week 12, % CD4+ cells was higher in dogs fed 5 and 10 mg lutein whereas the CD8+ population was not influenced by the diet (FIG. 7). On the other hand, dogs fed 20 mg lutein had significantly higher % CD8+ T cytotoxic cells on week 8 compared to control. The ratio of CD4:CD8 was similar among treatments on weeks 0, 4 and 8 but tended to be higher (P<0.08) in dogs fed 10 mg (2.5 0±0.16) compared to control (2.10±0.16).

On weeks 4 and 8, dogs fed lutein generally had higher percentages of CD5+ cells than unsupplemented controls and was statistically significant with dogs fed 5 and 20 mg lutein (FIG. 8). Dogs fed 20 mg lutein also had elevated % MHC class 11 cell populations on week 8 and 12 compared to unsupplemented dogs (FIG. 8). Dogs fed lower amount of lutein had MHC class 11 populations similar to control.

In contrast to other lymphocyte subpopulations, dietary lutein did not significantly influence the CD21+ B cell population.

### Interleukin-2 Production

The production of IL-2 by PHA-stimulated PBMC in whole blood cultures did not differ significantly among dietary treatments throughout the experimental period. Concentrations of IL-2 in culture medium averaged 15.7±0.4 ng/mL throughout the study.

### Immunoglobulin Production

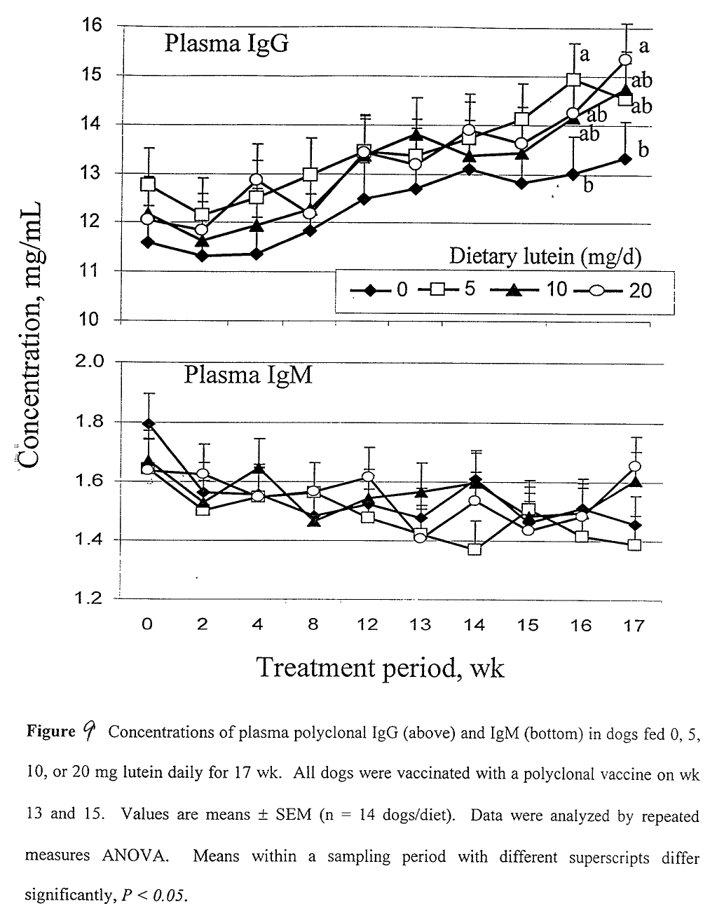

Plasma IgG concentrations tended to increase (P>0.05) throughout the 17-week sampling period as illustrated in FIG. 9. Concentrations were similar among dietary treatments during the first 12 weeks of dietary supplementation. However, after revaccination on week 15, plasma IgG was higher (P<0.05) on week 16 in dogs fed 5 mg lutein (P<0.05) and on week 17 in dogs fed 20 mg lutein whereas the concentration of plasma IgM was not changed (FIG. 9).

### Lipid Peroxidation

Lutein supplementation did not significantly affect plasma TBARS activity which averaged 95.8±0.1 nmol MDA/L across all treatments and periods.

### Discussion

The results show that dietary lutein significantly enhances cell-mediated immune response in the dog. Lutein supplementation stimulated the proliferative response of PBMC to PHA, Con A and PWM. There was a marked increase in PBMC proliferation in response to PHA and Con A at week 12 in dogs fed 20 mg lutein.

The data shows that enhanced mitogenesis by dietary lutein is likely attributed to increased population of lymphocytes. Dogs supplemented with lutein had higher populations of CD5+ and CD4+ cells. Dietary lutein may specifically act on T lymphocytes because no changes in B cell population were observed with lutein supplementation.

The data also showed that lutein supplementation significantly increased PWM-induced PBMC proliferation at weeks 8 and 12.

Dietary lutein also significantly increased the DTH response to vaccine, which is indicative of a specific immune response. While not wishing to be bound by any particular theory, this heighted DTH response may be explained through an increased population of MHC class II cells responsible for antigen processing and presentation. Lutein supplementation significantly increased the number of cells stained positive for MHC class II molecules compared to unsupplemented dogs.

Lutein did not significantly affect polyclonal antibody (IgG and IgM) production ex vivo in the canine during the first 12 weeks of supplementation. However, upon reexposure to the antigen, plasma IgG concentrations increased in lutein fed dogs, suggesting an anamnestic effect of dietary lutein in increasing the memory B cells' ability to secrete IgG.

In summary, dietary lutein enhanced canine T-helper cell population and the expression of MHC class II molecules, resulting in increased mitogen-induced canine PBMC proliferation and DTH response. Also, lutein is believed to increase Ig production.

### EXAMPLE 2

### Lutein Uptake in Cats

Fifty-six female Tabby cats (10 months old) were randomly assigned to be fed 0, 1, 5, or 10 mg lutein daily for 12 weeks. The lutein contained 76.66% lutein and 5.23% zeaxanthin and was incorporated into the basal diet (see Table 3) to a final concentration of 250 mg lutein/kg of diet.

TABLE 3

| Component | Composition (g/kg) |
|---|---|
| Moisture | 72 |
| Protein | 310 |
| Ash | 52 |
| Fat | 217 |
| Crude fiber | 14 |
| Calcium | 11.5 |
| Phosphorus | 8.3 |
| Gross Energy (kcal/kg) | 5412 |

Prior to being fed to the cats, the lutein-containing diet was mixed with additional basal diet to achieve the desired lutein concentration. The final diets were mixed weekly and stored at −20° C. in sealed containers undernitrogen gas. Prepared food analyzed periodically showed no significant destruction of lutein. Food and water were available ad libitum. Cats were housed (7 cats/pen; 1.5×5.0 m) in a temperature (20 to 22° C.) and light (14 h light) controlled facility.

Blood was collected by jugular venipuncture into heparinized evacuated tubes on weeks 0, 2, 4, 8 and 12 and aliquots used for HPLC analysis and for assessing immune responses.

### Extraction of Plasma for HPLC

Plasma lutein, retinol and α-tocopherol were analyzed by reverse phase HPLC. Briefly, plasma protein was precipitated with an equal volume of ethanol containing 0.1% butylated hydroxytoluene (BHT) (Aldrich Chemical Co., Milwaukee, Wis.). The mixture was extracted with 5 mi of a 1:1 mixture of petroleum ether:anhydrous diethyl ether and the dried residue resuspended in mobile phase. The mobile phase was a mixture of acetonitrile, methanol and chloroform (47:47:6, v/v/v). The internal standard used was retinyl acetate (Sigma Chem. Co., St. Louis, Mo.). Samples were eluted on a 5 μm C18 reverse phase column (3.9×150 mm; Resolve, Millipore Corp., Milford Mass.) with a flow rate of 1.0 ml/min. The identity of the eluted compounds was verified by comparing their absorption spectra with those of standard compounds.

### Delayed Type Hypersensitivity (DTH)

The DTH response was evaluated on week 0, 6, and 12 after the initiation of lutein feeding. Cats were mildly sedated with an i.m. injection of ketamine hydrochloride (5 mg/kg; Fort Dodge, Iowa) and acepromazine (0.1 mg/kg; Vedco, St. Joseph, Mo.). The hair on the flank was clipped and the area wiped with 70% ethyl alcohol. All cats were injected i.d. with 100 μl of saline (8.5 g/L; control), concanvalin A (Con A; 0.5 g/L) to measure nonspecific immunity, and with an attenuated polyvalent vaccine (Felocell, Pfizer, NY, N.Y.) containing feline herpesvirus-1, feline calicivirus, feline parvovirus and Chlamydia psittaci to measure specific immunity. The DTH response was assessed by measuring skin induration at 24, 48 and 72 h after injection with the aid of a digital micrometer (Mitsutoyo, Tokyo, Japan). The response was expressed as a percentage of skin thickness measured at the same injection site at 0 h.

### Peripheral Blood Mononuclear Cell Proliferation

Whole blood was used to analyze the mitogenic proliferative responsiveness of peripheral blood mononuclear cells (PBMC) to phytohemagglutinin (PHA), Con A and pokeweed mitogen (PWM) in order to mimic in vivo conditions. Heparinized whole blood was diluted 1:12 with RPMI-1640 containing 25 mM of Hepes, penicillin (100 U/mL) and streptomycin (100 μg/mL) (Sigma, St. Louis, Mo.) (hereafter termed complete medium). Preliminary studies using undiluted blood and blood diluted 1:2, 1:4, 1:8, 1:12 and 1:16 showed optimal repeatability and response to mitogens with the 1:12 dilution. The mitogens were used at the following final concentrations previously determined to provide both maximal (the higher mitogen concentration) and suboptimal (the lower mitogen concentration) proliferation: PHA (1.25 and 0.25 μg/mL), Con A (2.5 and 0.5 μg/mL) or PWM (0.125 and 0.025 μg/mL). Tritiated thymidine uptake was measured by liquid scintillation and PBMC proliferation response was expressed as cpm of stimulated cultures corrected for cpm of unstimulated cultures.

### Lymphocyte Subpopulations

Blood leukocytes were separated on Histopaque-1119 (Sigma, St. Louis, Mo.) and cells washed three times with phosphate-buffered saline (PBS, pH 7.4). Contaminating RBC were lysed with NH₄Cl (8.4 g/L) and cell viability determined by trypan blue exclusion. Lymphocyte subsets were determined by FACScan flow cytometry (Becton Dickinson, San Jose, Calif.) immediately after cell preparation. Isolated PBMC were resuspended to 1×10⁷ cells/mL phosphate buffered saline (PBS) supplemented with 2% gamma globulin-free serum, 5% goat serum and 0.2 g/L sodium azide (Baker, Phillipsburg, N.J.). For immunofluorescence analysis, 5×10⁵ cells were incubated for 30 minutes on ice with a previously determined optimal concentration of mouse anti-cat primary monoclonal antibody. The antibodies were specific for the following lymphocyte subsets: total T cells (anti-CD3), T-helper cells (anti-CD4), T cytotoxic/suppressor cells (anti-CD8), lymphocytes expressing major histocompatibility complex (MHC) class II antigens (anti-MHC class II), and B cells (anti-CD21). Cells were then washed three times and incubated with a secondary antibody, fluorescein isothiocyanate (FITC)-conjugated goat F(ab′)₂ anti-mouse IgG (H+L) (Caltag, Burlingame, Calif.) for 30 minutes on ice to visualize the bound antibody. Stained cells were fixed in 4% paraformaldehyde in preparation for acquisition. Appropriate negative controls were included to correct the level of background fluorescence. Fluorescence analysis was performed on lymphocytes gated by forward and side scatter. Data were expressed as a percentage of positive-staining cells corrected for cells stained nonspecifically with the secondary antibody.

### NK Cytotoxicity Assay

The Crandell feline kidney fibroblast was used as the target cell. Target cells were resuspended to 2×10⁵ cells/mL and triplicate 100 μL aliquots were pipetted into a 96-well U-bottom plate (Costar, Cambridge, Mass.) and cells allowed to adhere. Isolated PBMC (effector cells) were then added to the target cells to achieve effector:target cell (E:T) ratios of 25:1, 12.5:1 and 6.25:1. The cell mixture was incubated for 8 hours after which 100 μL Rose Bengal (2.5 g/L in PBS; Sigma, St. Louis, Mo.) were added and incubated at room temperature for 5 minutes. Optical density was measured at 550 nm using an ELISA plate reader. The percentage of NK-specific lysis was calculated as follows:

Specific cytotoxicity (%)=100×{1−[(OD of target cells and effector cells−OD of effector cells)/(OD of target cells)]}

### IL-2 Production

Whole blood was diluted 1:2 in RPMI-1640 complete medium and cultured for 48 hours in the presence of 5 μg/mL Con A. The conditioned medium was removed and frozen at −80° C. The IL-2 concentrations were determined in duplicate by ELISA using a human IL-2 kit (Intergen, Purchase, N.Y). The human anti-IL-2 was previously determined to cross-react with feline IL-2.

### Serum IgG

Serum IgG concentration was determined by single radial immunodiffusion (SRID). Goat antiserum to cat IgG (15%, whole molecule) (Sigma, St. Louis, Mo.) was incorporated into agarose (10 g/L in PBS; Sigma, St. Louis, Mo.). The loading volume for the test serum and standard was 5 μL and the plates were incubated for 24 hours in a humidified chamber. Ring diameters were measured using a SRID reader (Transidyne General Corp., Ann Arbor, Mich.).

### Lipid Peroxidation (TBARS)

Lipid peroxidation was assessed by the TBARS (thiobarbituric acid-reactive substances) assay on plasma collected on weeks 0, 2, 4, 8 and 12. Duplicate 500 μL of cat plasma was boiled for 45 minutes with 3 mL of phosphoric acid (10 g/L) and 1 mL of TBA (6 g/L). After cooling, the TBA-MDA (malondialdehyde) complex was extracted with 4 mL of n-butanol. The butanol layer was separated by centrifugation (1,000×g for 10 minutes) and absorbance read (Beckman, Fullerton, Calif.) at 532 nm. The absorbance unit was converted to nmoles MDA from a standard curve generated with a 1,1,3,3,-tetramethoxypropane (TMP) (Sigma, St. Louis, Mo.).

### Statistics

Data were analyzed by split-plot ANOVA using the General Linear Model of SAS (Statistical Analysis System). The statistical model was Yᵢⱼₖ=μ+Treatment₁+Catⱼ(Treatment₁) (subplot error term to be used to test the effects of treatment)+Sampling periodₖ+Treatmentᵢ*Periodₖ+eᵢⱼₖ. Differences between treatment means within a sampling period were compared using orthogonal contrast. Differences with P<0.05 were considered as statistically significant.

### RESULTS

### Body Weight and Food Intake

Dietary lutein did not significantly affect changes in body weight during the experimental period (data not shown). Body weight across all diets and periods averaged 1.41±0.04 kg. Average daily food intake was 83.2 g across the study period and was not significantly different among treatments.

### Plasma Lutein

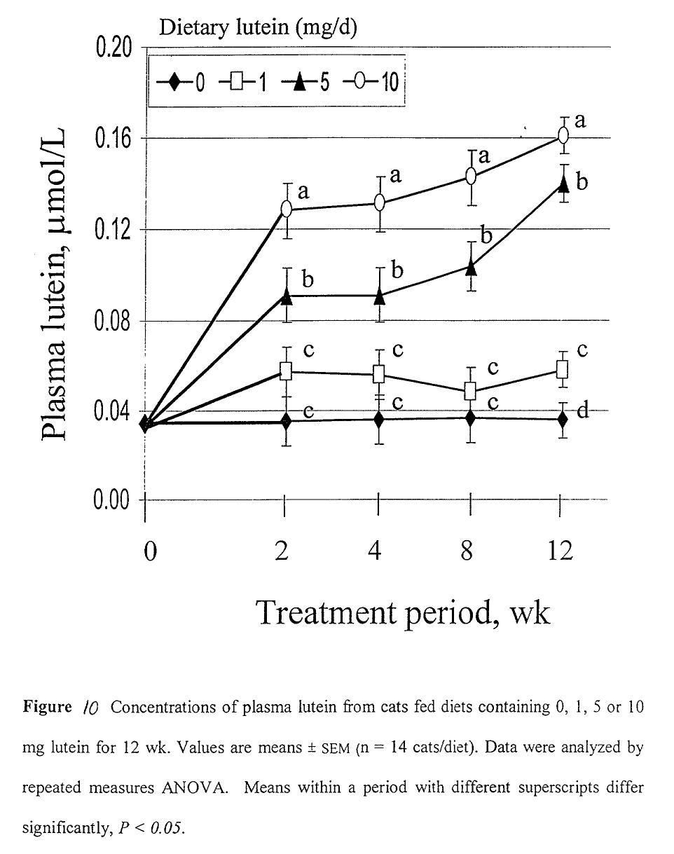

Prior to lutein supplementation, the baseline plasma lutein concentration averaged 0.036±0.005 μmol/L in all cats. Lutein concentrations in unsupplemented cats remained at this concentration throughout the study period. Plasma lutein increased in a dose-dependent manner between week 2 and 12 as shown in FIG. 10. Lutein concentrations in lutein-fed cats increased rapidly on week 2 and concentrations continued to increase, albeit more gradually, through week 12 in cats fed 5 or 10 mg lutein. Lutein supplementation did not influence the concentration of plasma retinol and α-tocopherol which averaged 0.56±0.03 and 14.9±0.9 μmol/L, respectively.

### Mitogen-Induced PBMC Proliferation

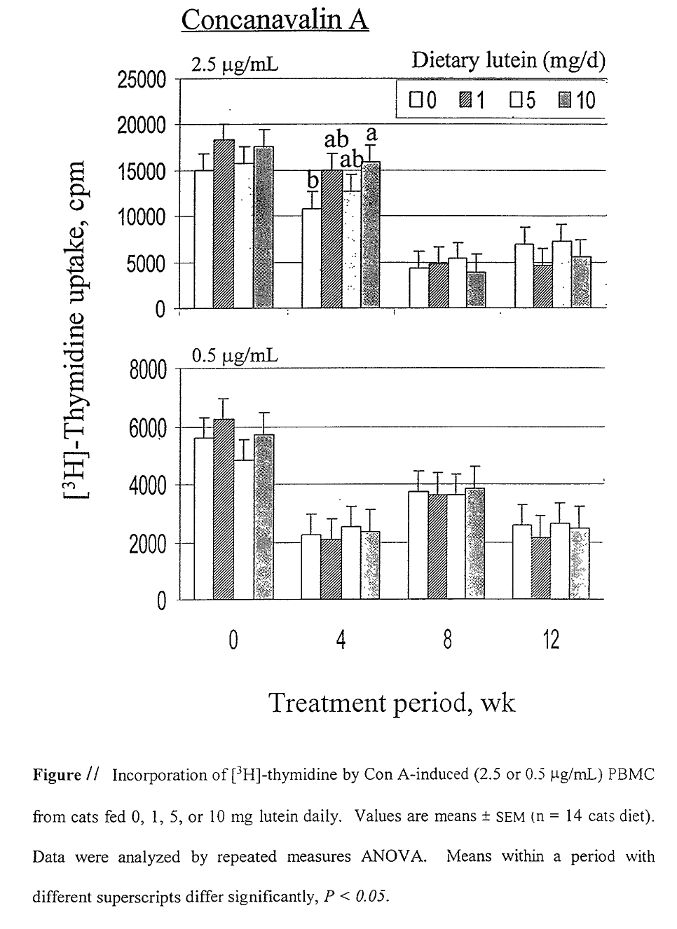

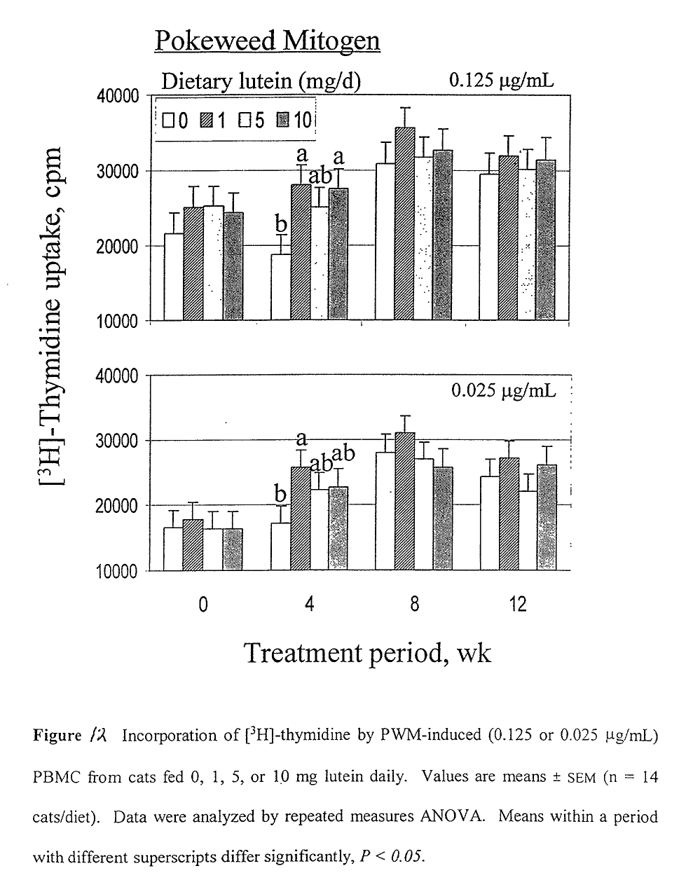

Dietary lutein did not significantly influence spontaneous proliferation by unstimulated PBMC and averaged 1,744±147 cpm throughout the study. Cats fed 10 mg lutein showed significantly higher PBMC proliferative response to Con A (2.5 μg/mL) at week 4 but treatment differences were not observed on week 8 and 12 (see FIG. 11). No significant treatment difference was observed with a suboptimal dose (0.5 μg/mL) of Con A. There were no significant dietary effects on PHA-induced PBMC proliferation throughout the study. Tritiated thymidine uptake with 1.25 and 0.25 μg/mL PHA averaged 6,464±504 and 1,934±147 cpm, respectively. Of the 3 mitogens used, PWM produced the highest proliferation (FIG. 12). Lutein supplemented generally increased PBMC proliferation on week 4; differences were significant when cultures were stimulated with 0.125 μg/mL (1 and 10 mg lutein) and 0.025 μg/mL (1 mg lutein) of PWM.

### Delayed-Type Hypersensitivity (DTH)

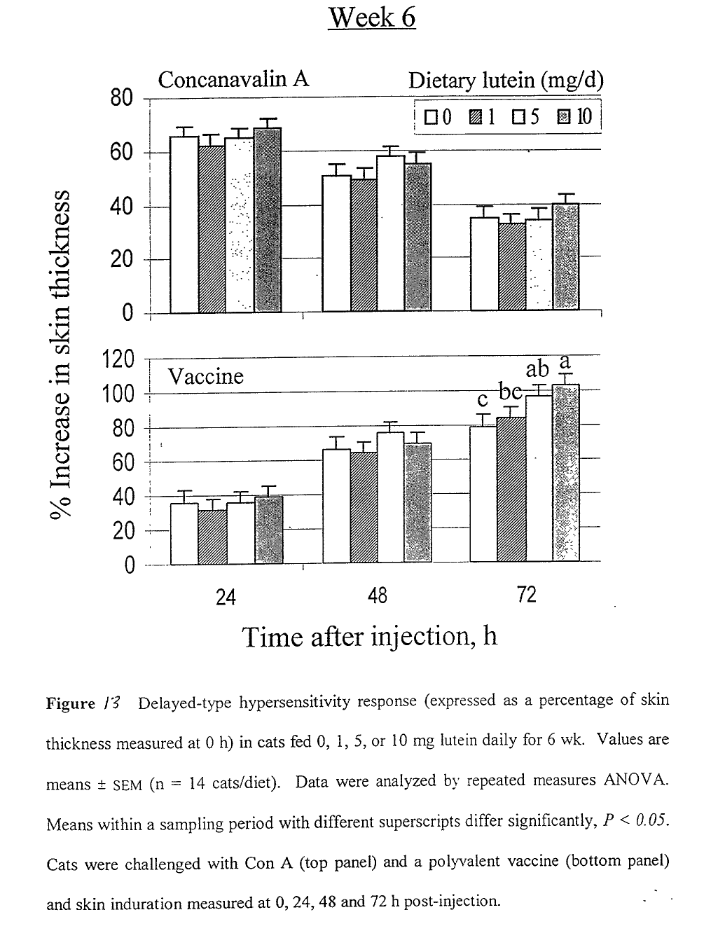

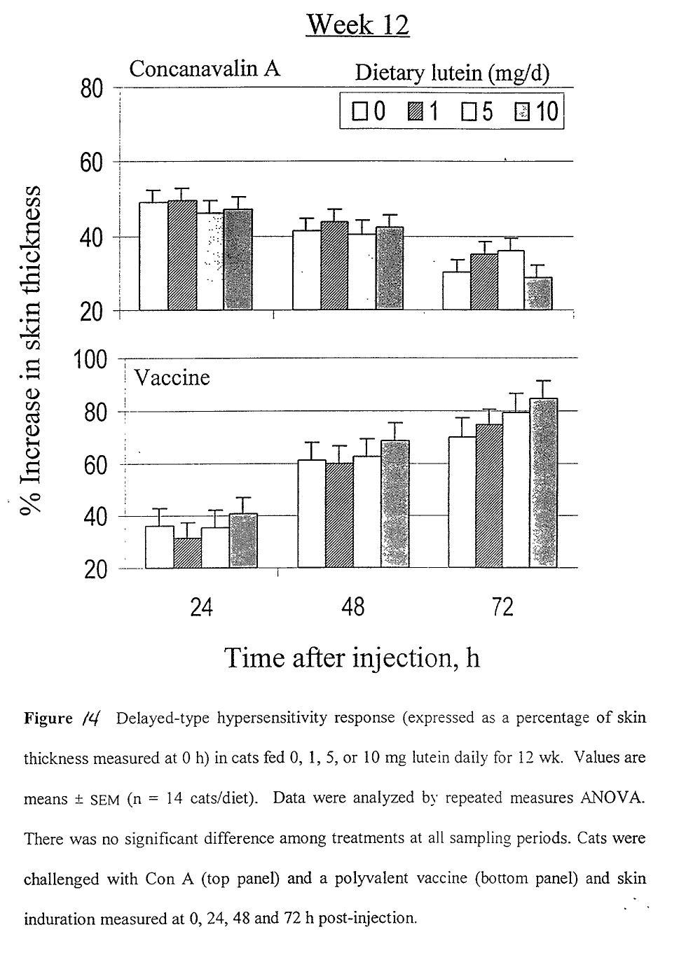

Prior to lutein supplementation, no significant differences were observed in DTH response among dietary treatments (data not shown). The DTH response to saline was low (averaged 4.3±0.6%) and not significantly different among treatments during all periods studied. On week 6, no significant dietary effect on DTH response to Con A was observed (FIG. 13). However, by week 6 of lutein feeding, there was a significant dose-related DTH response to vaccine when measured at 72 h post-injection; response was significantly higher with cats fed 5 or 10 mg lutein. By week 12, no significant treatment difference was observed with both Con A and vaccine (FIG. 14); however, the same general trend in a dose-related DTH response persisted with vaccine on week 12 at 72 h post-injection as was observed on week 6.

In general, peak DTH response to Con A was observed around 24 h post-injection whereas maximal response to vaccine occurred between 48 and 72 h.

### Natural Killer (NK) Cell Activity

Cytotoxicity activity of NK cells at effector:target cell (E:T) ratios of 6.25:1, 12.5:1 and 25:1 are illustrated in Table 4.

TABLE 4
Natural killer (NK) cell activity (% specific lysis) of isolated
PBMC in cats fed daily with 0, 1, 5, or 10 mg lutein¹
Dietary lutein (mg/d)

| | | | | |
|---|---|---|---|---|
| Week | 0 | 1 | 5 | 10 |

Effector: Target ratio of 6.25:1

| | | | | |
|---|---|---|---|---|
| 0 | 18.4 ± 6.1^{a} | 19.8 ± 5.5^{a} | 17.1 ± 5.8^{a} | 22.8 ± 6.5^{a} |
| 4 | 42.8 ± 4.8^{ab} | 46.6 ± 4.6^{a} | 41.2 ± 5.0^{ab} | 33.2 ± 4.8^{a} |
| 8 | 24.9 ± 5.3^{b} | 28.0 ± 4.8^{ab} | 34.3 ± 5.0^{ab} | 41.1 ± 5.5^{a} |
| 12 | 26.2 ± 5.0^{b} | 45.8 ± 5.5^{a} | 37.4 ± 5.8^{ab} | 39.7 ± 6.1^{ab} |

Effector: Target ratio of 12.5:1

| | | | | |
|---|---|---|---|---|
| 0 | 28.5 ± 5.8^{a} | 30.0 ± 7.0^{a} | 20.8 ± 7.0^{a} | 20.7 ± 8.2^{a} |
| 4 | 44.4 ± 5.3^{a} | 44.1 ± 4.9^{a} | 37.5 ± 5.1^{a} | 33.7 ± 5.1^{a} |
| 8 | 33.3 ± 6.4^{ab} | 27.4 ± 6.5^{b} | 44.4 ± 6.2^{ab} | 48.2 ± 6.5^{a} |
| 12 | 33.8 ± 6.4^{a} | 40.5 ± 6.1^{a} | 35.9 ± 5.8^{a} | 38.2 ± 7.0^{a} |

Effector: Target ratio of 25:1

| | | | | |
|---|---|---|---|---|
| 0 | 27.6 ± 6.1^{ab} | 21.2 ± 6.2^{ab} | 17.2 ± 6.3^{b} | 38.8 ± 8.3^{a} |
| 4 | 39.5 ± 5.2^{a} | 36.2 ± 4.4^{a} | 45.0 ± 4.9^{a} | 33.9 ± 4.6^{a} |
| 8 | 32.2 ± 6.7^{c} | 44.9 ± 8.0^{ab} | 32.6 ± 5.3^{bc} | 47.8 ± 5.5^{a} |
| 12 | 34.0 ± 10.2^{a} | 35.6 ± 6.2^{a} | 26.9 ± 6.3^{a} | 29.2 ± 9.5^{a} |

¹Values are presented as means ± SEM (n = 14/diet/period). Data were analyzed by repeated measures ANOVA. Means within a period with different superscripts differ significantly, P < 0.05

No significant treatment difference in cytotoxicity was observed on week 0 prior to lutein supplementation. However, on week 8, NK cell cytotoxic activity was higher in cats fed 10 mg lutein; differences were significant with E:T ratios of 6.25:1 and 25:1. Also, cats fed 1 mg lutein also showed significantly higher cytotoxicity on week 12 (E:T of 6.25:1) and 8 (E:T of 25:1). No significant treatment differences were observed with E:T ratio of 12.5:1 even though the same general trend was evident.

### Lymphocyte Subpopulations

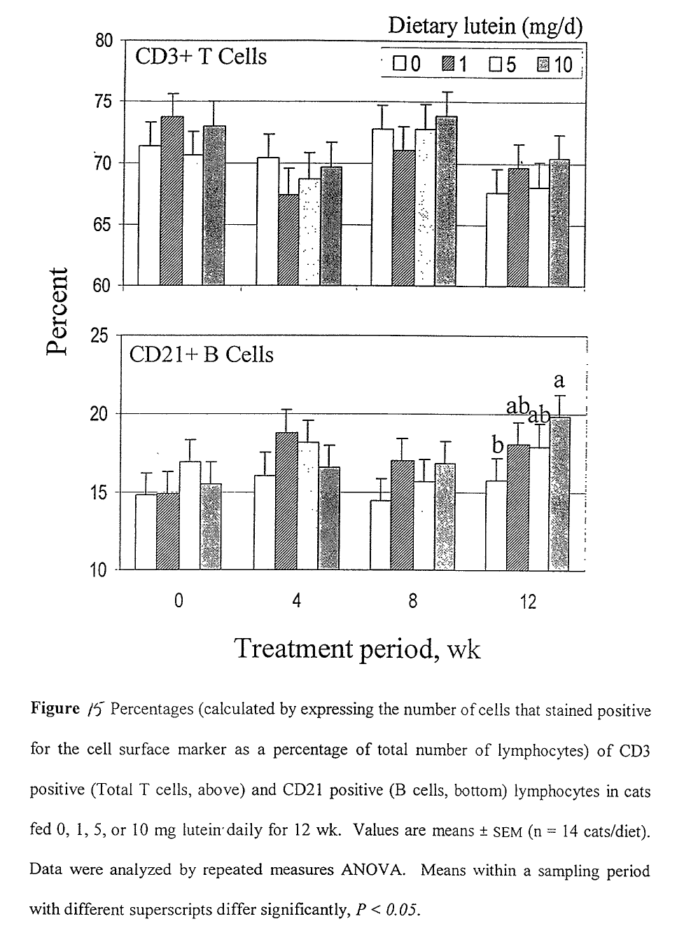

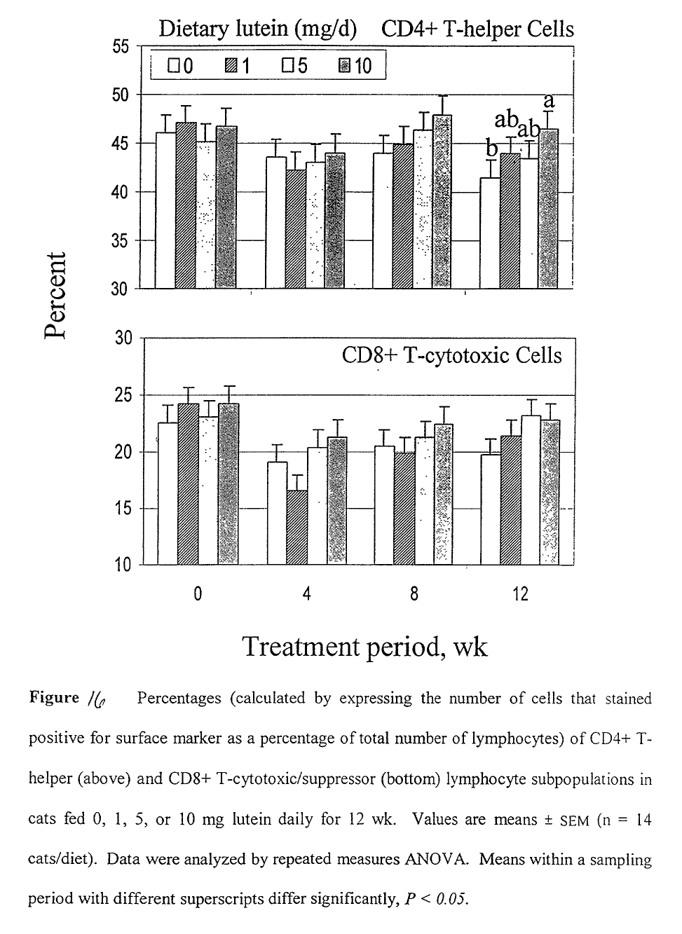

Dietary lutein did not influence significantly the total number of T cells (CD3) (FIG. 15) However, on week 12 of supplementation, the percentage of CD21 + B cells was significantly elevated in cats fed 10 mg lutein. Changes in the CD4+ T helper cell population (FIG. 16 were similar to that of the CD21+ cells. In contrast, the percentage of CD8+ T cytotoxic cells (FIG. 10), the ratio of CD4:CD8, and the percentage of MHC class II+cells were not influenced significantly by dietary lutein in any of the time periods studied.

### Interleukin-2 Production

There were no significant differences in IL-2 production throughout the experimental period. The average concentration of IL-2 in Con A-stimulated cultures throughout the study was 15.1±0.79 ng/mL.

### Antibody Production

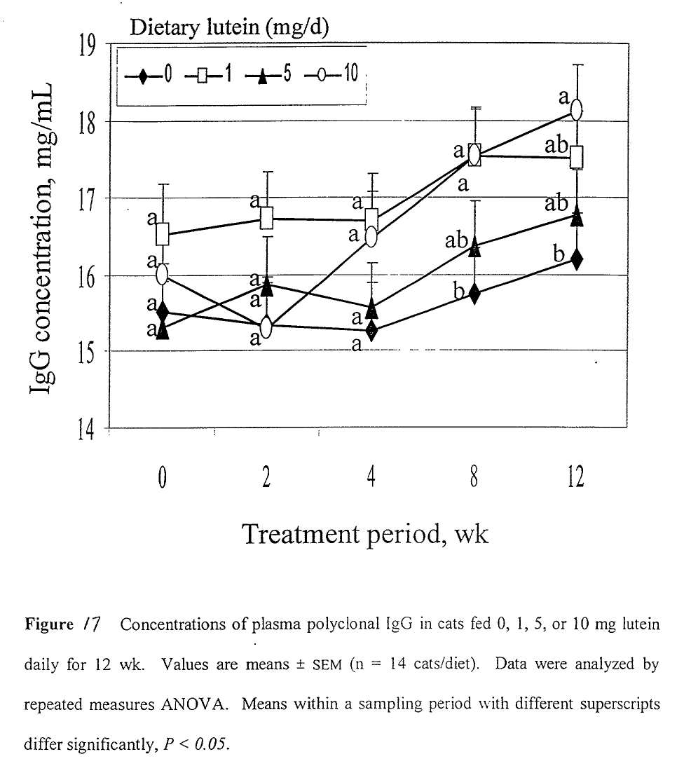

Plasma IgG concentration was not significantly different at the commencement of the study and averaged 15.85±0.62 mg/mL (FIG. 17). However, by week 8 of lutein feeding, cats fed 1 or 10 mg lutein had significantly higher concentrations of plasma IgG. This trend persisted through week 12 where plasma IgG in cats fed 10 mg lutein remained significantly higher than unsupplemented animals.

### Lipid Peroxidation

Dietary lutein did not significantly influence plasma TBARS activity and concentrations averaged 20.3±0.2 nmol MDA/L across all diet groups and periods.

### Discussion

The results showed that lutein modulates immune response in the domestic cat. Cats absorb lutein into the blood and the lutein is subsequently taken up by circulating leukocytes. The results showed that the absorption of lutein is dose- and time-dependent and does not significantly alter plasma concentrations of retinol and α-tocopherol.

Dietary lutein stimulated the proliferative response of PBMC to Con A and PWM. The enhanced lymphocyte proliferation by dietary lutein is most likely mediated via the alteration of cell surface marker expression on lymphocytes.

Dietary lutein also significantly increased the B and T-helper cell populations. NK cell cytotoxic activity was also enhanced by dietary lutein. Lutein is believed to upregulate NK cell activity through the activation of Th cells or its direct action on NK cells.

Dietary lutein significantly increased DTH response to vaccine in a dose-related manner at week 6. However, there was no significant boosting effect with repeated inoculation of the antigens. Even though no marked changes in total T cell population were observed, lutein supplementation significantly increased the CD4+ T helper cell population but not the CD8+ T cytotoxic cells. This increase in T helper cells may explain the enhanced DTH response observed in lutein-supplemented cats.

Cats fed lutein also showed significantly higher serum IgG concentrations. The observed enhancing effect of lutein on antibody production is believed to be mediated through the action of the T helper cells.

In summary, the data indicates that dietary lutein is an important immunomodulating micronutrient in the domestic cat.

While certain representative embodiments and details have been shown for purposes of illustrating the invention, it will be apparent to those skilled in the art that various changes in the methods and apparatus disclosed herein may be made without departing from the scope of the invention, which is defined in the appended claims.

What is claimed is:

1. A process for enhancing immune response and improving the overall health of a companion animal comprising the step of feeding said animal a diet containing an effective amount of lutein for a time sufficient for said lutein to be absorbed by said animal.

2. A process claimed in claim 1 in which said diet includes from about 1 to about 50 mg/day of lutein.

3. A process as claimed in claim 1 in which said diet includes from about 2 to about 315 mg lutein per kg of diet.

4. A process as claimed in claim 1 wherein said companion animal is a dog.

5. A process as claimed in claim 4 in which said diet includes from about 5 to about 20 mg/day of lutein.

6. A process as claimed in claim 1 wherein said companion animal is a cat.

7. A process as claimed in claim 6 in which said diet includes from about 5 to about 10 mg/day of lutein.

8. A process as claimed in claim 1 in which said diet comprises about 20 to 40% crude protein, about 4 to 30% fat, and about 4 to 20% total dietary fiber.

9. A process for increasing the lutein concentration in the circulating blood of a companion animal comprising the step of feeding said animal a diet containing an effective amount of lutein for a time sufficient for said lutein to be absorbed into the bloodstream of said animal.

10. A process for increasing IgG concentrations in a companion animal comprising the step of feeding said animal a diet containing an effective amount of lutein for a time sufficient for said lutein to be absorbed into the bloodstream of said animal.

11. A process for increasing lymphocyte cells in a companion animal comprising the step of feeding said animal a diet containing an effective amount of lutein for a time sufficient for said lutein to be absorbed into the bloodstream of said animal.

12. A supplement for enhancing immune response and improving the overall health of a companion animal comprising, on a dry matter basis, from about 0.001 to about 2% by weight lutein.

13. The supplement as claimed in claim 12 wherein said companion animal is a dog.

14. The supplement as claimed in claim 13 comprising from about 0.01% to about 1% by weight lutein.

15. The supplement as claimed in claim 12 wherein said companion animal is a cat.

16. The supplement as claimed in claim 15 comprising from about 0.01% to about 1% by weight lutein.
